(19) United States
(12) Patent Application Publication
(10) Pub. No.: US 2017/0215880 A1
(43) Pub. Date: Aug. 3, 2017

(54) SURGICAL STAPLER ADAPTABLE DEVICE, USEFUL FOR SLEEVE GASTRECTOMY OR TOTAL GASTRECTOMY SURGERIES

(71) Applicant: Cristóbal Alfredo DAVANZO CASTILLO, Santiago (CL)

(72) Inventor: Cristóbal Alfredo DAVANZO CASTILLO, Santiago (CL)

(21) Appl. No.: 15/386,589

(22) Filed: Dec. 21, 2016

(30) Foreign Application Priority Data
Jan. 28, 2016 (CL) 220-2016

(51) Int. Cl. A61B 17/072; A61F 5/00

(52) U.S. Cl. CPC A61B 17/07207; A61F 5/0089; A61B 2017/07228; A61B 2017/07285; A61B 2017/07221; A61B 2017/07271; A61B 2017/07242; A61B 2017/00818

(57) ABSTRACT

A device that may be used as a surgical stapler comprises five rows of staples towards the segment one wishes to suture and one row towards the segment that is extracted. In a back area it comprises two sections separated in the middle for a knife to pass through. At least one of the sections presents a curve-shaped end to adapt it to the stapler.

### STATE OF THE ART

Mechanical sutures or staplers have changed the perspective of surgery, allowing the execution of procedures that are less invasive and with better results. Particularly, in the field of gastrointestinal surgery, mechanical sutures have permitted the execution of multiple procedures that today are routinely executed in a minimally invasive manner. Even so, this has not been exempt of adverse effects, where the most outstanding are two main complications related to mechanical sutures: the filtration and bleeding of the cut line.

Multiple improvements have been introduced since the creation of mechanical sutures, which have reduced the rate of bleeding and filtration, but there is still a significant percentage of patients that suffer this type of potentially fatal complications. The filtration rate accepted internationally for morbid obesity surgery programs is of up to 5%.

On the other hand, there is an increase in bariatric surgery procedures associated to the increased growth of obesity, and above all, of one of the newer techniques, gastric sleeve surgery, that has become one of the surgical procedures against obesity that is used most. The sleeve gastrectomy is a surgery indicated for patients with severe or morbid obesity and is currently one of the techniques used the most to achieve weight loss surgically. It consists of the skeletization of the major gastric curvature and then the tubing of the stomach from close to the pylorus to the angle of His. This tubing is executed using surgical staplers that section, and at the same time seal both sides with staples, achieving the decrease of its volume. An effect of reduction of hunger is achieved simultaneously because of the resection of the body and gastric antrum that reduces the production of the hunger hormone or Ghrelin.

The section and suture of the stomach is the origin of most of the complications related to the bleeding and to the filtration of the suture line, but none have achieved a significant reduction of these complications. The highest rate of complications is found in the first days after the operation; therefore, it is highly probable that the mechanical factor is of great importance in its origin. The filtrations in this type of surgery are more difficult to treat than in other bariatric surgical procedures. There are different strategies for handling this complication, that go from the installation of an endoscopic prosthesis to the gastrectomy and Roux-en-Y reconstruction. There is no consensus as to which is better or the algorithm indicated.

On the other hand, the physiopathology of the postsurgical gastric filtration has not been well defined; it has been qualified as a multifactorial event in which the quality of the tissues, the metabolic state of the patient, surgical technique and failure of the medical supplies used, all converge. It appears mainly within the first week after surgery and is generally located in the upper segment of the cut/suture area, close to the angle of His. Even improving the technical and patient-related factors, the filtration rate continues being a problem that is present nowadays, proposing a constant challenge to the medical industry for the development of devices that reduce this complication even more or eliminate it directly.

Some documents related to the technology are detailed below:
1.Patent U.S. Pat. No. 5,782,396 (Mastri et al.) titled “Surgical stapler”. The innovation divulges a surgical stapling apparatus that is useful for applying sequentially parallel lines of surgical fasteners to the body tissue, and optionally, cut the tissue held in laparoscopic surgical procedures. This device can be employed with different disposable loading units.
2. Patent application US 2010/0282816 (Scirica et al.) titled “Stapler cartridge and channel interlock”. The technology protects a surgical stapling instrument that consists of an anvil, a cartridge set that defines a longitudinal axle and that has a cartridge channel, which has a distal end with at least one projection that extends transversally to the longitudinal axis, it also includes a cartridge of staples located within the cartridge channel. This instrument can be used in association with other lineal stapling devices known both in endoscopic as well as open construction.
3. Patent ES 2393015 (Marczyk et al.) titled “Retaining systems of separable reinforcement material for use with a surgical stapling device”. A surgical stapler is protected to display staples in a tissue, that includes: a pair of jaws to engage with the tissue, including the pair of jaws, a cartridge of staples and an anvil, at least one of the jaws defining a plurality of cavities, a reinforcement material of the row of staples, a plurality of retainers, where the latter are equivalent to a staple that has an arch and a pair of feet that extend from the arch.
4. Patent U.S. Pat. No. 5,911,353 (Bolanos et al.) titled “Disposable loading unit for surgical stapler”. The patent mentions a disposable loading unit for use together with a surgical stapling device, set up for the sequential application of a plurality of surgical staples in the body tissue. The loading unit includes a staple cartridge body that has a longitudinal path that extends through it and a plurality of separated retention slots, a plurality of closure ejection elements, and an actuator supported within the body of the staple cartridge.

The above-mentioned technologies do not ensure that the filtration and bleeding of the cut line will be avoided completely in gastric sleeve surgeries or total gastrectomy; therefore, there is a need to develop new medical instruments.

### DESCRIPTION OF THE INVENTION

The current technology corresponds to a device adaptable to surgical staplers, useful for gastric sleeve surgeries or total gastrectomy. This device, of the stapler cartridge type, is equipped with five rows of staples towards the segment one wishes to suture and one row towards the segment that is extracted. This layout of the rows of staples permits a union of tissues with a high safety profile, which advantageously reduces the complications in procedures that involve sutures of only one end of a segment of the digestive system, such as, for example, the filtrations.

This cartridge of staples, as it has five suture lines on the side of the remaining stomach, permits, on the one hand, a reduction in the bleeding while at the same time contributes greater mechanical support to the suture, which especially in the first days, must withstand situations of maximum stress due to the nausea and/or vomiting after the operation. This reinforcement, consisting of a greater number of rows of staples, provides greater strength which translates into support for the first lines of staples that might fail. On the other hand, the segment of stomach that is extracted must only be sutured by a line of staples, which is not very relevant as it is extracted and even its intraoperative rupture would not cause negative consequences for the patient. With regard to the bleeding, the mechanical reinforcement functions in a similar manner because having a longer segment of staples there is a greater possibility of closing smaller vessels independently of their tortuosity.

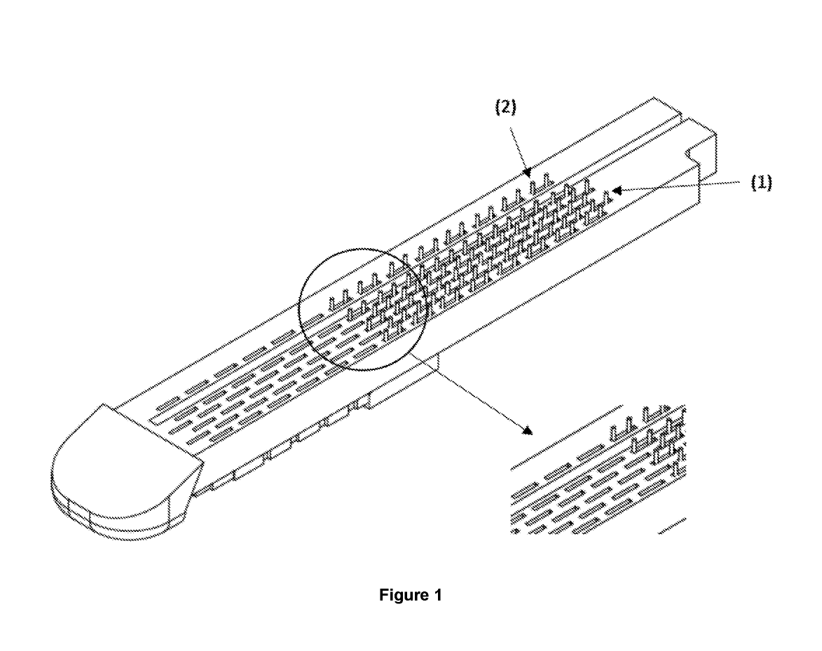

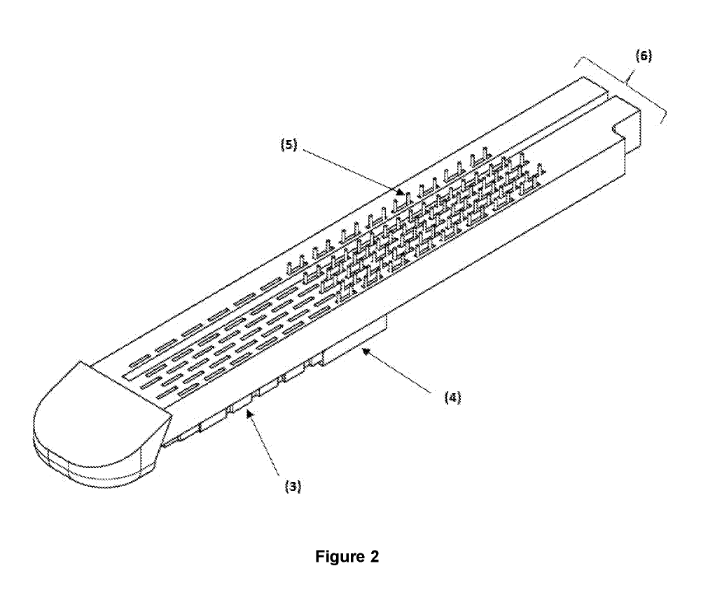

For a better understanding of the technology, FIGS. 1 and 2 are taken as reference, where an upper and lower view of the cartridge is shown, respectively. In (1) the five rows of staples can be appreciated and in (2) a row of staples; (3) corresponds to the staple pushers which, depending on the device, can go in the cartridge or in the stapler that shoots them; (4) is a sliding actuator that, also depending on the device, can go in the cartridge or in the stapler that shoots them, which has the function of sliding to enter into contact with the staple pushers to start to apply the staples (5) sequentially; and (6) corresponds to the rear end made up of two sections, separated in the middle to allow a knife to pass through, in charge of making the incision. At least one section presents a curved end to permit the adaptation of the cartridge to a traditional stapler. The width of the cartridge is 10 mm and the length may vary between 4, 6 or 8 cm to adapt easily to any stapler.

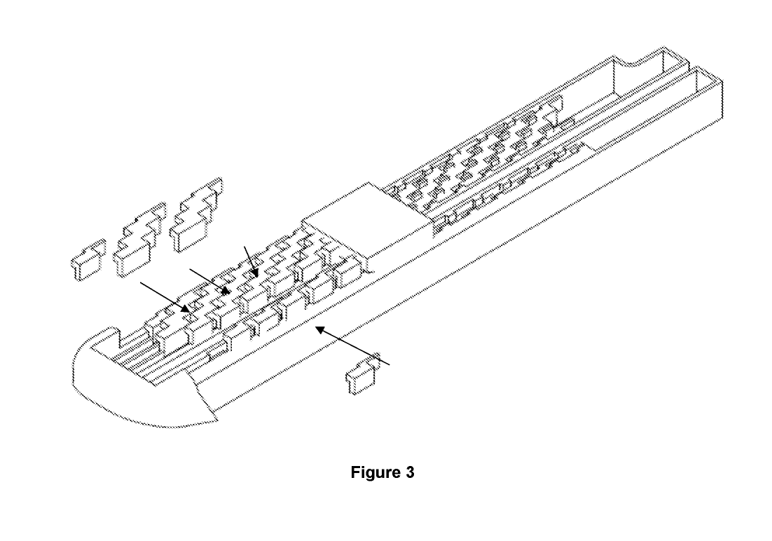

FIG. 3 shows an enlarged view of the staple pushers present in the cartridge, which permit the application of the staple and are associated to the slots of the cartridge. They are placed in contact sequentially and depending on their location in the cartridge can present shorter or longer sections, the latter for the case of the area that shoots five lines of cartridges. These pushers allow three different sizes of staples to be pushed, where the highest are the two lines to the left of the cut, that is equivalent to 4 mm high staples, then the two following of 3.5 mm and the ends that are on both sides of the incision that correspond to staples of 3 mm. The size of the staples may vary according to the thickness of the tissue to be sutured.

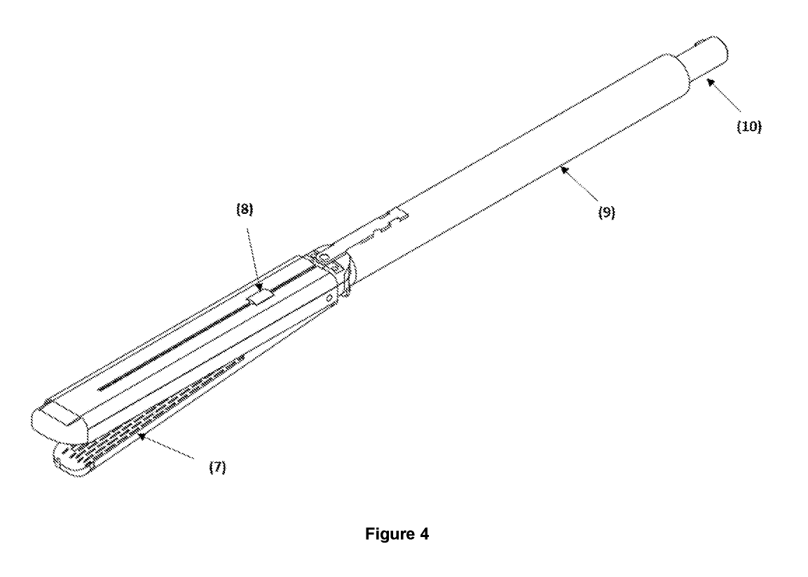

FIG. 4 presents a sketch of the conventional stapler, to which the cartridge of staples (7) is adapted with the arrangement of 5:1 lines of staples, that advantageously avoids the filtration and bleeding of the cut line in surgery. Furthermore, the location of the knife is shown in (8), in (9) the support of the knife and in (10) a mobility bar an axial movement control bar that transmits the stapler's longitudinal drive forces for the control of the axial movement of the head.

### EXAMPLE OF APPLICATION

The cartridge of staples was adapted to the staples most used universally (Endo GIA® and Echelon®) to verify its functioning on live animal tissue.

As an example of the operation carried out with both staplers conditioned with the cartridge of staples, in FIG. 2 we can see the cartridge of staples, where in its front part the staple pushers (3) have already been applied, while in the back the low section of the staples (5) that had already been applied is shown and in the upper section the pushers that were still in their original position.

It can be verified in situ that when five lines of staples are applied, an increase in the mechanical resistance and a significantly smaller bleeding of the cut line were achieved in both staplers.

The increase in the suture's resistance was verified when attempting to separate the joined tissue with a tension device that included weight measurement. The significant reduction of the bleeding was observed in a comparative study with the usual stapler, where this layout of 5:1 did not show any bleeding whatsoever on the cut line after its use. The above meant a significant improvement in the use of this device for this type of surgery.

1. A device adaptable to a surgical stapler CHARACTERIZED in that it consists of five rows of staples (1) towards the segment one wishes to suture and one row (2) towards the segment that is extracted, and in the back area (6) it presents two sections separated in the middle for a knife to pass through and where at least one of these sections presents a curve-shaped end to adapt it to the stapler.

2. A device adaptable to a surgical stapler according to claim 1 CHARACTERIZED in that the device presents a width of 10 mm and a variable length of 4, 6 or 8 cm.
